(19) United States
(12) Patent Application Publication
(10) Pub. No.: US 2006/0106402 A1
(43) Pub. Date: May 18, 2006

(54) Endovascular surgical method

(76) Inventor: Bruce McLucas, (US)

Correspondence Address: Seldon & Scillieri, 18th Floor, 10940 Wilshire Blvd., Los Angeles, CA, 90024-3952, US

(21) Appl. No.: 10/992,279

(22) Filed: Nov. 18, 2004

(51) Int. Cl. A61F 11/00

(52) U.S. Cl. 606/108

(57) ABSTRACT

An endovascular surgical method comprises the steps of (1) incising a patient to gain access to the interior of a selected blood vessel, (2) inserting a cannula through an aperture formed in a sealing member comprising an elastomeric membrane having an aperture for receiving the cannula, an elongated wall portion surrounding the aperture, an outer flange region, an expandable bellowed intermediate portion between the flange region and the elongated wall portion, and a layer of adhesive on the bottom surface of the flange region, (3) orienting the membrane so that the bottom surface of the flange region contacts the patient's skin when the cannula is inserted through the incision, the aperture and cannula being relatively sized to form a tight fit and seal around the cannula, and (4) perform surgery from within the selected blood vessel by inserting and withdrawing surgical instruments through the cannula to so that the intermediate membrane portion resiliently supports the cannula for movement against its elongated wall portion without transmitting stress to the flange portion that would cause the flange portion to pull away from the patient's skin.

### FIELD OF THE INVENTION

This invention relates to endovascular surgery methods.

### BACKGROUND OF THE INVENTION

Vascular surgery is surgery that involves a blood vessel; i.e., an artery or vein. When the surgery is done from within the blood vessel, it is called endovascular surgery.

Endovascular surgery is a procedure for treating two major problems that can develop in blood vessels: aneurysms (i.e., a weakness in a major blood vessel that causes a portion of the vessel wall to balloon out) and occlusions (i.e., a narrowing)

Endovascular surgery uses an easily accessible smaller artery to reach the problem and is typically performed without general anesthesia. During most endovascular procedures, a long plastic tube called a cannula is placed into the femoral artery in the groin. Using X-ray imaging, a physician advances the cannula to the aneurysm or narrowing.

To repair an aneurysm, a hollow, manufactured tube (a graft with metal attachments) is pushed through the cannula to the aneurysm and anchored in place. To open a narrowing, an angioplasty is performed using a balloon and stent wherein the balloon is inserted and advanced through the cannula to the narrowing and inflated. The stent (a small, mesh-like stainless steel tube) is then pushed through the cannula to the narrowing. The balloon is deflated and removed, and the stent expands to press against the inner walls of the artery and keeping it open.

Accordingly, a number of instruments such as wires, other catheters and the above-described instruments are pushed into, and subsequently withdrawn from, the cannula during various stages of the surgical procedure. During the withdrawal of the instruments, there is a risk that the cannula will unintentionally be withdrawn from the site, and even from the incision.

### SUMMARY OF THE INVENTION

The invention herein is an endovascular surgical method comprising the steps of: (a) inserting a cannula through an aperture in a membrane having an elongated wall portion surrounding the aperture, an outer flange portion, and an expandable bellowed intermediate portion therebetween, the membrane being oriented so that a sealing surface formed on at least a portion of the outer flange portion will face the patient as the downstream end of the cannula is inserted though an incision in the patient; (b) incising the patient to gain access to the interior of the Femoral artery, (c) inserting the downstream end of the cannula into the Femoral artery, (d) adhering the sealing surface of the membrane to the patient's skin circumscribing the incision and (e) passing instruments into and out of the cannula through said aperture while the membrane is sealed to the patient's skin, the relative sizes of the cannula and aperture being selected to form a tight fit and seal between the elongated wall portion and the cannula and when to prevent unintentional withdrawal of the cannula from the incision, and the intermediate portion of providing flexibility of cannula movement about one a vertical center line without imposing a stress that dislodged as the flange portion from the patient's skin. Further details concerning the invention will be appreciated from the following detailed description of the invention, of which the drawing is a part.

### DESCRIPTION OF THE DRAWING

FIG. 1 is a perspective view of a properly positioned cannula and sealing member utilized in accordance with the invention;

FIG. 2 is a perspective view, partially in section, of a sealing member used in accordance with the invention; and

FIG. 3 is a cross sectional view of a cannula supported within the sealing member in accordance with the invention.

### DETAILED DESCRIPTION OF THE INVENTION

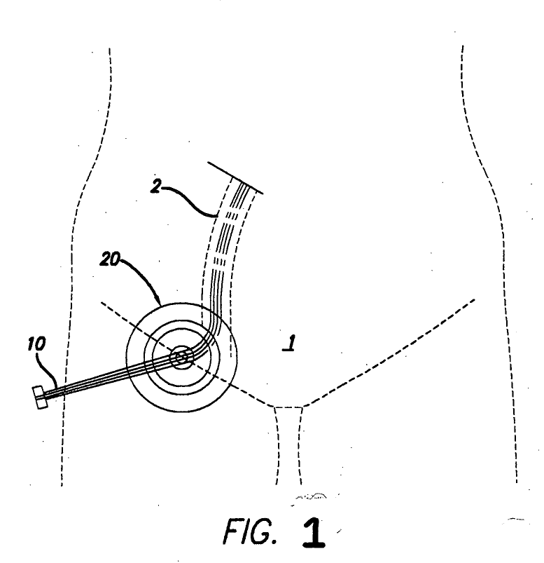

FIG. 1 is a perspective view of properly positioned cannula and sealing member utilized in accordance with the invention. Entry into the arterial system, whether into coronary arteries or a peripheral artery, is typically through the groin region 1 via the Femoral artery 2 on the patient's right side. A 5 or 6 French cannula 10 is inserted through an incision overlying the Femoral artery. The cannula acts as an introducing sleeve for other catheters and wires during the subsequent procedure.

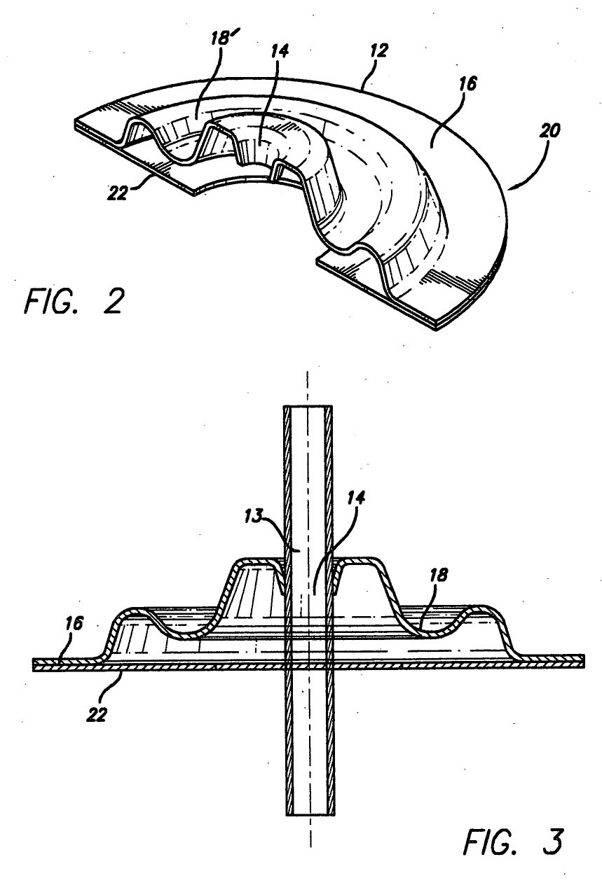

The cannula may slide out when catheters, wires and the like are removed. Accordingly, a sealing device 20 is inserted over the cannula prior to the cannula's insertion through the incision. The sealing device is best illustrated in FIG. 2 and is similar in structure to a trochar support shown in my U.S. Pat. No. 5,073,169, issued Dec. 17, 1991, the contents of which are hereby incorporated by reference. Briefly, the sealing member 20 comprises a membrane 12 having an aperture 14 to accommodate the insertion of the cannula. The membrane includes an outer flange portion 16 at its outer perimeter, and an intermediate bellows-like portion 18 between the flange and elongated wall portions. An adhesive layer 22 is applied at the undersurface of the flange portion 16.

The sealing member is approximately 15 mm in diameter, with a center aperture of approximately 2 mm. The adhesive is preferably placed around the outer 5 mm, more or less, of the 15 mm diameter.

The sealing member itself is a thin, flexible membrane formed from a material such as molded thermoplastic elastomer. The material must be sufficiently flexible to conform to the shape of the underlying body part and to adhere to creases in the patient's skin.

As illustrated in FIG. 3, the cannula 10 is inserted through the aperture 14 of the membrane, and then through the incision to the desired depth. The sealing member is then slid down the cannula until it's a piece of-coated bottom surface contacts the patient's skin. The sealing member is then pressed against the patient's contacted skin to form a seal. To ensure that a fresh and clean adhesive layer is presented against the patient's skin, the adhesive coating is preferably covered with a removable protective liner, such as a paper liner, that is removed prior to contact with the patient's skin.

As the surgical procedure progresses, catheters and wires may be slid in and out of the cannula as needed without the risk of inadvertently withdrawing the cannula from the incision. The relative sizes of the aperture and cannula are such that the walls of the aperture fit tightly about the cannula, substantially preventing its inadvertent withdrawal.

In addition, the bellows-like portion 18 of the sealing member permits a certain degree of angular movement from vertical. (As used herein, the term “vertical” means the direction substantially normal to the plane of the patient's incision.) Because the cannula is resiliently supported by the walls of the aperture and, consequently, by the bellows-like portion 18, the cannula can pressed against the walls of the aperture 14 without transmitting stress to the flange portion 16 that would cause the flange portion to pull away from the patient's skin. Any such pressure exerted against the walls of the aperture causes the bellows-like portion 18 to flex without straining the adhesive bond between the sealing member and the patient's skin. As pressure against the aperture wall is relieved by a countermovement of the cannula, the bellows-portion 18 returns to its normal position as the pressure against it is released, so that the sealing member continues to firmly support the cannula in a substantially vertical orientation.

Although the present invention and its advantages have been described in detail, it should be understood that various changes, substitutions and alterations can be made herein without departing from the spirit and scope of the invention as defined by the appended claims.

I claim:

1. An endovascular surgical method comprising the steps of:
incising a patient to gain access to the interior of a selected blood vessel;
inserting a cannula through an aperture formed in a sealing member comprising an elastomeric membrane having an aperture for receiving the cannula, the membrane having an elongated wall portion surrounding the aperture, an outer flange region and an expandable bellowed intermediate portion between the flange region and the elongated wall portion, said sealing member having a layer of adhesive on the bottom surface of the flange region and being oriented so that the bottom surface of the flange region contacts the patient's skin when the cannula is inserted through the incision, the aperture and cannula being relatively sized to form a tight fit and seal around the cannula,
perform surgery from within the selected blood vessel by inserting and withdrawing surgical instruments through the cannula to so that the intermediate portion resiliently supports the cannula for movement against elongated wall portion without transmitting stress to the flange portion of the sealing member that would cause the flange portion to pull away from the patient's skin.

2. An endovascular surgical method comprising the step of:
inserting a cannula into the incision in a patient via an elastomeric sealing member having a cannula-accommodating aperture sized to form a tight fit and seal around the cannula, an elongated wall portion surrounding the aperture, an outer flange region, an expandable bellowed intermediate portion between the flange region and the elongated wall portion, and a layer of adhesive formed on the bottom surface of the flange region to contact the patient's skin generally circumscribing the incision when the cannula is inserted;
pressing the adhesive surface against the patient's skin generally circumscribing the incision to seal the member to the patient;
inserting and withdrawing surgical instruments through the cannula to perform surgery from within the selected blood vessel so that the intermediate portion resiliently supports the cannula for movement against elongated wall portion without transmitting stress to the flange portion of the sealing member that would cause the flange portion to pull away from the patient's skin; and
removing the sealing member from the patient's skin near the completion of the surgical procedure.
